(19) United States
(12) Patent Application Publication
(10) Pub. No.: US 2025/0380971 A1
(43) Pub. Date: Dec. 18, 2025

(54) OFFSET ROD CONNECTOR

(71) Applicants: Oluwatodimu Richard RAJI, San Francisco, CA (US); Dimitriy Kondrashov, Sherman Oaks, CA (US); Jeremi Leasure, Carlsbad, CA (US)

(72) Inventors: Oluwatodimu Richard RAJI, San Francisco, CA (US); Dimitriy Kondrashov, Sherman Oaks, CA (US); Jeremi Leasure, Carlsbad, CA (US)

(21) Appl. No.: 19/242,108

(22) Filed: Jun. 18, 2025

Related U.S. Application Data
(60) Provisional application No. 63/661,441, filed on Jun. 18, 2024.

(51) Int. Cl. A61B 17/70; A61B 17/00; A61B 17/56

(52) U.S. Cl. CPC A61B 17/7049; A61B 17/7002; A61B 17/7083; A61B 2017/00477; A61B 2017/564

(57) ABSTRACT

Provided are rod connector systems, kits and methods that include a main body defining a first side opening configured for insertion of a primary rod therein, and a tulip defining a second side opening configured for insertion of a secondary rod therein, the tulip being inserted into a top opening in the main body and attached to the main body above the main body, in which the main body includes a locking mechanism for retaining the primary rod at a desired position therein, and the tulip includes a locking cap for maintaining the secondary rod at a desired position therein.

### CROSS REFERENCE TO RELATED APPLICATION

This application claims the benefit of U.S. Provisional Application 63/661,441 filed on Jun. 18, 2024, the contents of which are hereby incorporated herein by reference in their entirety

### FIELD

The present application relates generally to offset rod connector devices and systems that may be used in spinal surgery and methods of treating a patient using the present offset rod connector devices and systems. Example embodiments include offset rod connector devices, systems and methods for the minimally invasive, percutaneous insertion and placement of offset rod connectors within spinal rod fixation constructs.

### BACKGROUND

The evolution of minimally invasive surgical (MIS) techniques has significantly impacted the field of spinal surgery, aiming to reduce operative trauma and enhance patient recovery. Advantages of minimally invasive spine surgery include reduced surgical trauma, accelerated patient recovery, and a diminished incidence of postoperative complications such as infections. These benefits represent a significant advancement in surgical practices and patient care outcomes.

Indications for the placement of spinal rods can include: backing up a long construct after multilevel XLIF, ALIF, or ACDF surgery, treatment of spinal fractures with or without neurological deficits, fixation of unstable 3-column Chance fractures, and fixation of unstable, or ankylosed spine in patients with ankylosing spondylitis, diffuse idiopathic skeletal hyperostosis or ankylosis due to advanced degenerative disc disease.

The use of percutaneous placement of pedicle screws and rods, in spine surgery has grown in recent years due to the reduced physiological trauma to the patient during the operation, faster recovery times, and reduced chance of post-op infection. One limitation of percutaneous fixation is the inability to place a cross-link or a domino device, which connect two rods together.

Usage of connectors is widespread in open thoracolumbar fusion, and is especially useful for long constructs and compromised bone due to osteopenia or osteoporosis. Placing a parallel connector/domino creates a multiple rod construct and decreases the chance of rod breakage by increasing rigidity, and placing a cross-link—a transverse connector—creates a fixed angle construct and decreases the chance of pullout of the screws—especially at the cephalad and caudal ends of the long construct. The inability to place such a connector in a percutaneous construct is a significant limitation and can lead to construct failure due to screw pullout, lessened rigidity of the construct, especially in highly unstable spine fractures.

Domino connectors are spinal fixation devices that connect spinal fusion rods that are approximately parallel to each other, for example, in revision cases or when multiple rod constructs are used. In revision cases, they may allow for connecting new rods to old ones without completely removing existing implants. Domino connectors are crucial in linking adjacent rods to enhance the structural integrity and longevity of spinal rod fixation systems, particularly in complex reconstructions, fracture and tumor fixation or multilevel deformity surgery involving spinal osteotomy. However, the traditional design and installation process of domino and crosslink connectors, which necessitates a direct, horizontal or vertical insertion of the additional rod, are incompatible with the minimal access corridor afforded by percutaneous surgery. This limitation threatens the durability of the spinal construct and increases the risk of rod fracture under dynamic instability or mechanical stress.

Surgeons are thus often faced with a dilemma: to maintain the minimally invasive ethos at the potential expense of construct stability or to revert to more invasive methods for incorporating offset connectors. This challenge undermines the core advantages of minimally invasive surgery, necessitating a reevaluation of the surgical approach and, in some instances, compromising the benefits associated with reduced trauma and expedited recovery. It forces the surgeon to compromise the strength of the construct just to keep the surgery minimally invasive.

### SUMMARY

The present application addresses the problems associated with conventional devices and methods and provides improvements on these devices. The present application describes devices, systems, kits and methods for an offset top-loaded rod connector, which may be utilized for example, as an adjunct to existing open and percutaneous spinal rod fixation constructs. Devices and methods of the present application, include a retainer-portal assembly configured to attach on to an existing rod construct, after which a rod or plate can be installed into the slot to connect existing or additional rods to the construct. The rods may then be kept in place using a retention or locking cap. The present devices, systems and methods also provide a lateral offset for example, in a domino connector, allowing a more versatile use of the connector. The present devices, systems and methods may or may not require any additional incisions by a surgeon, apart from those already created.

Provided herein are rod connector devices and systems, including a main body defining a first side opening configured for insertion of a primary rod therein, and a tulip defining a second side opening configured for insertion of a secondary rod therein, the tulip being inserted into a top opening in the main body and attached to the main body above the main body, in which the main body includes a locking mechanism for retaining the primary rod at a desired position therein, and in which the tulip includes a locking cap for maintaining the secondary rod at a desired position therein. Additional devices and systems may include a locking mechanism to lock the primary rod in the main body and/or a locking cap to lock the secondary rod in the tulip. Further devices and systems may include a cannula detachably attached to the tulip.

Also provided herein are kits that include a main body and a tulip as set forth herein. Kits may also include a cannula or one or more tabs configured to form a cannula upon attachment to the tulip. Kits may also include at least one additional component including instructions for use, a primary rod and/or secondary rod, a locking mechanism, a locking cap, or a tool for assembly or insertion of the rod connector system or a component thereof.

Further provided are methods of connecting multiple spinal rods that include attaching a main body to a primary rod, attaching a locking mechanism to the main body, the locking mechanism being configured to lock the primary rod to a desired position with respect to the main body by compressing the primary rod, attaching a tulip to the main body, placing a secondary rod into an opening of the tulip, adjusting the tulip or the secondary rod, such that the secondary rod has a desired angle with respect to the primary rod, and driving a locking cap into the tulip to lock the secondary rod at a desired position.

### BRIEF DESCRIPTION OF THE DRAWINGS:

Non-limiting embodiments of the present application are demonstrated by the illustrative figures herein, which include the following:

FIGS. 1A, 1B and 1C depict an embodiment of a main body in accordance with non-limiting examples, from front, side and top views.

FIGS. 2A, 2B and 2C depict an embodiment of a main body in accordance with non-limiting examples.

FIGS. 3A and 3B depict an embodiment of a tulip in accordance with non-limiting examples.

FIG. 4 depicts a device or system including a main body, a tulip and a cannula in accordance with non-limiting examples.

FIGS. 5A and 5B depict a device or system including a main body and a tulip, as well as a cannula in accordance with non-limiting examples, showing rods positioned therein at angles parallel with respect to each other.

FIG. 6 depicts a device or system including a main body and a tulip, as well as a cannula in accordance with other non-limiting examples, showing rods positioned therein at offset angles with respect to each other.

FIG. 7 depicts a device or system including a main body and a tulip, as well as a cannula in accordance with other non-limiting examples, showing rods therein at different offset angles with respect to each other.

FIGS. 8A, 8B, 8C, and 8D depict an embodiment of a main body in accordance with non-limiting examples, from side (FIGS. 8A-8C) and top (FIG. D) views, having a tulip therein.

### DETAILED DESCRIPTION

Example embodiments provided herein include offset rod connector devices, systems and methods for the minimally invasive, percutaneous insertion and placement of offset rod connectors within spinal rod fixation constructs. Offset connectors may include for example, fixed angle (domino or cross-connector) and polyaxial (domino or crosslink) configurations.

While embodiments are described herein, it is to be understood that this disclosure is illustrative and exemplary and is made for the purposes of providing a full and enabling disclosure. The detailed disclosure herein of example embodiments is not intended, nor is to be construed, to be limiting. All illustrations of the figures are for the purpose of describing selected embodiments and are not intended to limit the scope of the present application.

As should be understood, any embodiment may incorporate only one or a plurality of the above-disclosed aspects of the disclosure and may further incorporate only one or a plurality of the above-disclosed features. Items described in the singular herein may be provided in plural, as can be seen, for example, in the drawings. Thus, the description of a single item that is provided in plural should be understood to be applicable to the remaining plurality of items unless context indicates otherwise.

Each term used herein refers to that which an ordinary artisan would understand such term to mean based on the contextual use of such term herein. To the extent the meaning of a term used herein—as understood by the ordinary artisan differs in any way from any particular dictionary definition of such term, the meaning of the term as understood by the ordinary artisan in the context of the present application, should prevail.

The terms “a” or “an”, as used herein, are defined as one or more than one unless the contextual use dictates otherwise. The term plurality, as used herein, is defined as two or more than two. The term another, as used herein, is defined as at least a second or more.

When used herein to join a list of items, “or” denotes “at least one of the items,” but does not exclude a plurality of items of the list.

Throughout the specification, when a component is described as “including” a particular element or group of elements, it is to be understood that the component is formed of only the element or the group of elements, or the element or group of elements may be combined with additional elements to form the component, unless the context indicates otherwise.

It will be understood that when an element is referred to as being “connected” or “coupled” to or “on” another element, it can be directly connected or coupled to or on the other element or intervening elements may be present.

Ordinal numbers such as “first,” “second,” “third,” “primary,” “secondary” etc. may be used simply as labels of certain elements, steps, etc., to distinguish such elements, steps, etc. from one another. Terms that are not described using “first,” “second,” etc., in the specification, may still be referred to as “first” or “second” in a claim. In addition, a term that is referenced with a particular ordinal number (e.g., “first” in a particular claim) may be referenced elsewhere without an ordinal number or with a different ordinal number (e.g., “second” in the specification or another claim).

Any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Spatially relative terms, such as “below,” “lower,” “above,” “upper,” “top,” “bottom,” “front,” “back,” and the like, may be used herein for ease of description to describe positional relationships, such as illustrated in the figures, for example. It will be understood that the spatially relative terms encompass different orientations of the device in addition to the orientation depicted in the figures.

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

The present application describes devices, systems and methods for both open and percutaneous top-loading cross-linking or domino-connecting as an adjunct to existing open and percutaneous spinal rod fixation constructs.

Devices or systems provided herein (also referred to as “connectors”) which may include a main body and a tulip, and optionally a tulip and/or other components) may join spinal rods of congruent or non-congruent diameters. They can be used for example in revision surgery, e.g. where a previous spinal fusion surgery has failed or complications arise, to connect new rods to existing hardware, which may minimize the extent of the revision surgery. The present connectors may also be employed in complex spinal reconstructions for adult deformities, for example, when using multiple rod constructs.

In alternative embodiments throughout this application, instead of a traditional circular rod, a different configuration rod may be used as well: semicircular, oblong, oval, rectangular, square, trapezoidal, etc. Thus, the term “rod” as used herein, is intended to encompass devices not only having a circular cross section, but also devices having other shaped cross-sections (semicircular, oval, rectangular, square, trapezoidal, etc), and other devices that may be elongated, such as plates, beams, rails, etc. Reference to a “rod” or plate is intended to include one or any of these configurations. Additionally, the term “primary rod”, “fixation rod” and “primary fixation rod” are used interchangeably herein. The terms “secondary rod”, “cross-connecting rod” and “secondary cross-connecting rod” are used interchangeably herein.

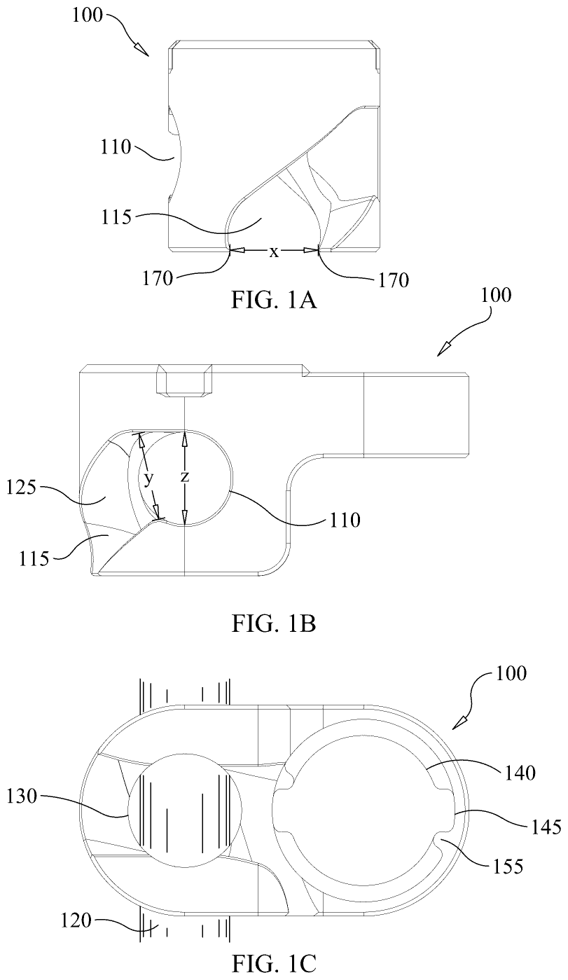

The present application provides a main body device. FIGS. 1A, 1B, and 1C depict a main body device 100 in accordance with example embodiments, from front, side and top views, respectively. The main body 100 is configured to define a first side opening 110 of the main body 100 configured to receive and/or retain a first or primary rod or plate. The terms first rod, first primary rod and first primary fixation rod are used interchangeably herein and are also intended to encompass a first plate.

FIGS. 1A and 1B show retention features to receive and retain a first primary rod on the retaining clamp. The present devices provide a rod attachment feature, which may include for example a start position opening 115 (defined by walls of the main body 100) of the primary rod 120 during installation of the primary rod 120 within the main body 100, and the first side opening 110 in the main body 100. The main body 100 may be configured to hook or clip or otherwise attach onto a primary fixation rod 120. It is noted that the first primary rod is not part of the present device itself. However one or more rods and/or plates may be included in systems or kits provided herein. The present devices, such as a main body 100, may also include a retention feature configured to hold the primary fixation rod 120 within and/or at a desired position in the first side opening 110 of the main body 100. The retention feature 125 may include for example, walls of a main body defining an opening 115, such as an undersized opening, having a width x or y relative to the diameter z of the first side opening 100 or relative to the diameter A of the primary rod 120, for retention of the primary rod 120 along a vertical axis of a tulip.

According to example embodiments the opening 115 in the main body 100 may be a helical cut out of the main body 100. A top portion of the helical cut out or opening 115 may have a width y, which may be an undersized opening relative to the diameter A of the primary rod, for example at initial and final positions of the primary rod. A bottom portion of the helical cut out or opening 115 may have a width x that may also be an undersized opening with respect to diameter A of the primary rod. These serve to provide tactile feedback and temporary resistance to removal during installation due to axial/compressive loads perpendicular to the primary rod at the initial opening and rotational torque loads at the final opening.

The present devices and systems may provide retention for the primary rod 120 by seating it after for example, a second temporary retention click and then locking it in place for example, with a locking mechanism 150, such as a set screw, which applies axial compression on the primary rod 120 perpendicular to the axis of the primary rod 120. A first top hole 130 in the main body 100 where the primary rod sits may be larger than the primary rod diameter x to allow for positioning of the clamp prior to locking. The locking mechanism or set screw 150 is inserted into the top of the clamp component or main body and tightened such that it compresses the primary rod onto the seat of the clamp, providing resistance against the primary rod 120.

As indicated above, according to example embodiments, widths x or y may be, or may not be undersized relative to width z of the first side opening 110 and another retention feature 125 may retain a primary rod or plate 120 within the first side opening 110. While a circular first side opening 110 or cut out is shown for example in FIG. 1B for first side opening 110, the first side opening 110 may be circular, elliptical, rectangular, polygonal, triangular or a combination of these shapes to be suitable for receiving a primary rod 120 or plate of varying shape. The first side opening 110 may also utilize multiple sized shapes to be adaptable to various rod sizes.

The retention feature 125 of the main body may be a portion of the main body 100 that is configured to define an opening that is contiguous with the first side opening 110, but is not configured in size or shape or position to allow a primary rod 120 within the first side opening 110 to fall or slip out of the first side opening 110. As indicated above, the retention feature 125 may define a helical opening 115. However other shapes and configurations are contemplated.

FIG. 1C shows a top view of the main body of FIGS. 1A and 1B, according to non-limiting example embodiments. Although FIG. 1C shows a primary rod 120 through the first side opening 110, the primary rod 120 is not part of the main body itself. As can be seen in FIG. 1C, the top of the main body may define a first top opening 130 and may define a second top opening 140. The first top opening 130 and/or second top opening 140 may be configured to receive a tulip 200, tool, or other accessory that may be used in the present systems and/or methods. In example embodiments, the first top opening 130 and/or second top opening 140 may be configured to assist in positioning (such as rotating) and/or locking a tulip 200 to a desired location.

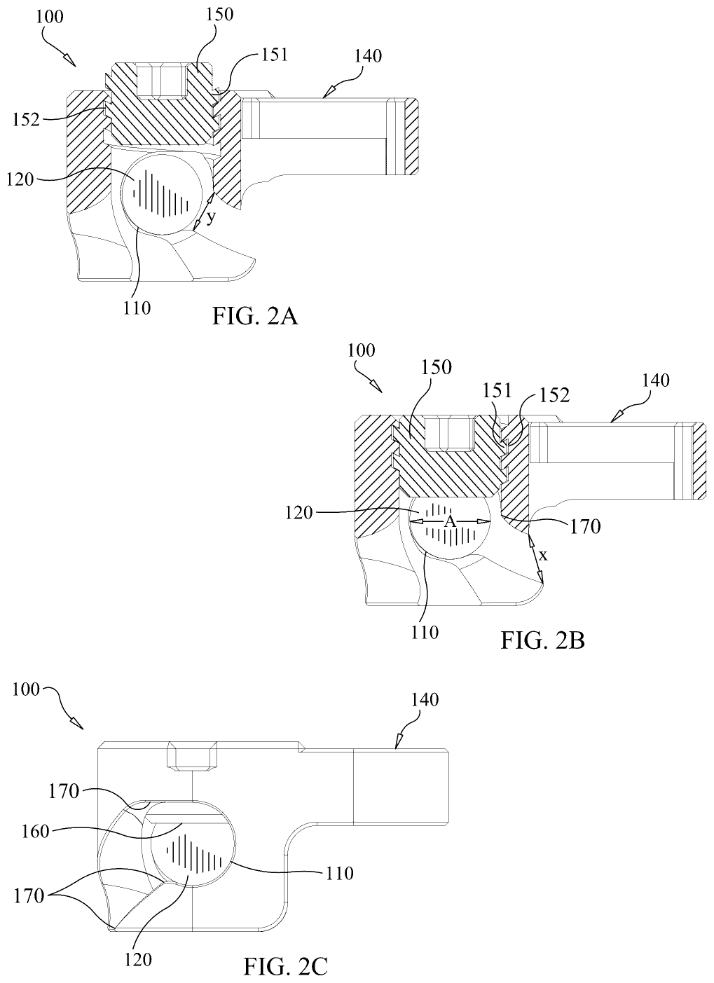

FIGS. 2A, 2B and 2C depict crosssectional side views (FIGS. 2A and 2B) and a side view (FIG. 2C) of a main body 100, respectively, having compression components for the primary fixation rod 120. In example embodiments, the main body 100 may include a locking mechanism 150, for example positioned within the first top opening 130 of the main body 100. In the depicted example, the locking mechanism 150 may be for example, a screw, as depicted in FIG. 2A at a partially unscrewed position, and in FIG. 2B at a more screwed in position. In FIG. 2A a top portion of the screw is above the top of the main body 100, and in FIG. 2B, the screw has been screwed further into the main body 100, which further locks or secures a primary rod 120 into a desired position in the main body 100. Although FIG. 2B depicts a top of the screw 150 being at the same height as the top of the main body, in example embodiments, they do not need to be perfectly aligned when the locking mechanism or screw 150 is engaged in the main body 100.

A top portion of the locking mechanism or screw 150 may be configured for example to recieve a screwdriver or other tool that may be used in positioning or rotating the screw 150 within the main body 100. In example embodiments, the screw may have male threads 151, which may be configured in size and pitch to engage with female threads in a first top opening 130 of the main body 100, such that the screw 150 may be screwed down into the main body.

The main body 100 may define a cut out(s) portion 115, that may be for example, helical in shape. The portions of the main body defining the cut out portion 115, may also feature one or a plurality of lips 170 for additional retention on the primary fixation rod 120. While a single circular first side opening 110 is shown for a rod having a circular cross section, multiple cut outs may be used to accommodate varying shapes and sizes of primary fixation rods. The first side openings or cut out(s) 110 may be circular, elliptical, rectangular, polygonal, triangular or a combination of these shapes. The cut out 110 may also utilize multiple sized shapes to be adaptable to various rod sizes.

The lip(s) 170 may serves to provisionally retain the primary rod 120 at a desired position. According to example embodiments, the lip(s) 170 may be configured such that they provide an audible and/or palpable confirmation to a surgeon of primary rod engagement at a desired location. Example embodiments may optionally include a relaxation slot at the end of the cut out 115, such as a helical cut out, defined by a retention feature 125 of the main body 100, to provide the ability for some elastic opening of the main body 100 to facilitate the capture of the primary rod 120 in the main body 100.

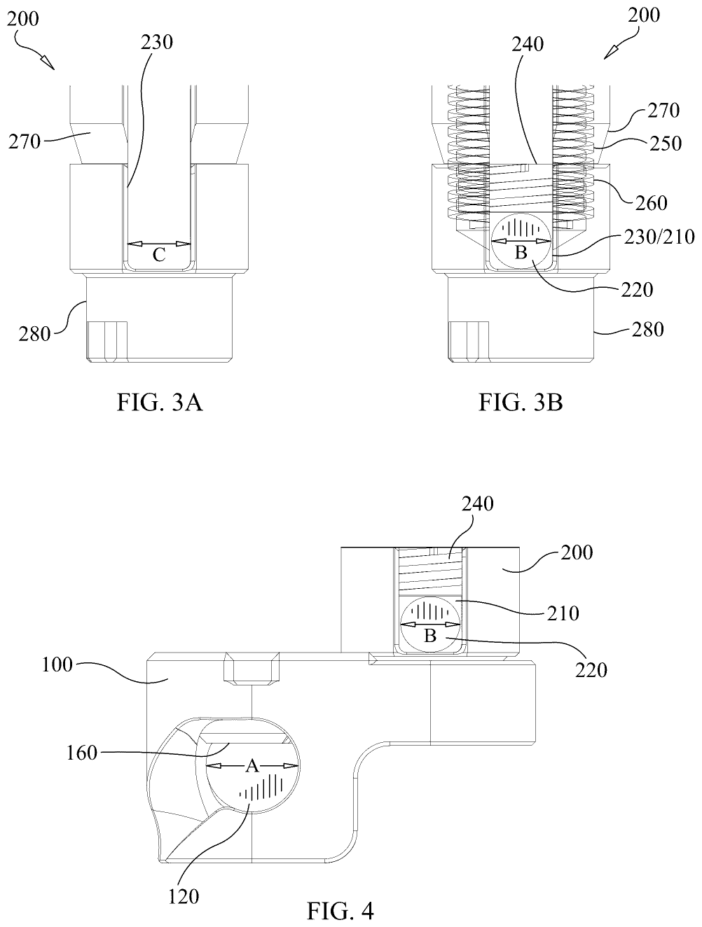

FIGS. 3A and 3B depict a tulip device 200 in accordance with non-limiting example embodiments. The tulip 200 may be configured for secondary rod 220 retention in the present devices and systems.

FIGS. 3A and 3B depict example representations of a slot 230 defined by internal walls of the tulip 200, which slot 230 may be configured and utilized as a seat for the secondary rod or plate 220. The slot 230 may be a second side opening 210 configured to receive a secondary rod or plate 220. An example slot 230 or side opening 210, as shown for example in FIGS. 3A and 3B, may have a width C larger than a width or diameter B of the secondary rod or plate 220, to allow for varying trajectories/variations of angulation of the secondary/cross-connecting rod or plate 220 about the principal/vertical axis of the tulip upon implantation of the present device into a patient. While a rectangular slot 230 profile is shown in FIG. 3A, according to other embodiments, the second side opening 210 or slot 230 profile may be circular, elliptical, rectangular, polygonal, triangular or a combination of these shapes. The slot 230 or second side opening 210 may also include one or more additional slots or openings (not shown) following the same and/or different trajectories configured to receive an additional rod, plate or tool for use with the present devices, systems or methods.

As shown in FIG. 3B, in example embodiments, above the opening 210, the tulip 200 may have a threaded feature 250 that is configured to interface with an optional threaded locking cap 240, such as a screw. In example embodiments, locking caps provided herein may not necessarily be threaded caps. The threaded locking cap 240 may be configured to be able to apply a radial and/or axial clamping force unto the secondary/cross-connecting rod or plate 220, for retention of the secondary rod or plate 220, upon an application of axial and/or torsional loads, through its driving feature(s). The driving features of the cap 240 is the portion of the cap 240 that a driver atttaches to. The cap can be designed to use different types of drivers (such as a screw driver, which may be a cross, hexagon, etc).

In example embodiments, the threaded feature 250 of the tulip 200 may be a female threaded feature 250 and the threaded locking cap 240 may have a male threaded feature 260 configured in size and shape to engage with the female threaded feature 250 of the tulip 200. Although threaded configurations for attachment of the locking cap 240 to the tulip 200, it is contemplated that a locking cap 240 may be attached to the tulip 200 by another configuration or method.

As shown in FIGS. 3A and 3B, in examples, the tulip 200 may include a top portion 270 or petals that expands outward at the top of the tulip 200. The geometry of the tulip 200 and the top portion/tulips 270 can be configured to facilitate alignment and engagement with corresponding device components, such as a cannula 300, for guiding the device components into a correct position for a secure connection.

The tulip 200 can be integrated with the main body 100 of example embodiments through a junction that maintains structural integrity under operational stresses. This integration may be designed to ensure that the tulip 200 can effectively transmit forces to the locking mechanism 150 housed within the main body 100 of example embodiments, enabling a secure and reliable connection. As part of the integration, a bottom portion 280 of the tulip 200 may be configured to be insertable into second top opening 140 of the main body 100.

A function of the tulip 200 can be to provide guided engagement with mating components. Its outwardly expanding petals 270 may be configured and shaped to guide the insertion of corresponding components, reducing the potential for misalignment and ensuring a precise connection. The tulip 200 design may provide self-aligning capabilities. As a mating component is introduced to a connector device of example embodiments, the petals 270 of the tulip 200 may flex to accommodate and guide the component into the optimal position for engagement, effectively compensating for minor alignment errors during the connection process.

Although petals 270 are depicted in some embodiments and not in others, it should be understood that petals 270 may be present or not in any of the embodiments herein. For example, FIG. 4 is a short tulip configuration that does not depict petals. A short tulip configuration without petals may be suitable for example for open surgery, in which tulips are not necessary. In other embodiments, for example in MIS methods, it may be useful or preferable that the tulip 200 may have one or more petals 270.

The main body member 100 may be configured such that it can attach to a primary rod, plate, etc. 120 from other manufacturers, such as rods or plates that may already be in a patient. The tulip 200 may be configured such that existing rods may be used as a secondary rod.

As shown for example in FIG. 4, devices and systems provided herein may include a main body 100 and a tulip 200 connected to the main body 100. The tulip 200 and the main body 100 may be connected to one another, permanently, or in a detachable manner. They may be made of the same or different materials.

The main body 100 and tulip 200 may also be configured to receive rods or plates having the same or different sizes, such as cross-sectional diameters. In example embodiments, the tulip and/or main body may be rotatable with respect to one another, such that the respective openings adapted to receive a first rod or first plate and a second rod or second plate are at a desired angle with respect to one another before or after insertion of one or both of the primary and secondary rods and/or plates 120 and 220. Rotation of the main body 100 and tulip 200 with respect to each other may occur before or after insertion of the present system into a patient. A mechanism or lock may be provided for fixing or locking the main body 100 and tulip 200 with respect to each other at a desired angle after it is achieved. In some embodiments, the main body and/or the tulip may include a locking mechanism, such as a bi-stable latch, a twist-lock system, or other lock, allowing for secure engagement and easy release of connected components. In example embodiments a bottom portion 280 of the tulip 200 may interface with the second top opening 140 of the main body 100 in a twist-and-lock fashion.

In alternative embodiments, the desired angle between the first side opening 110 of the main body 100 and the second side opening 210 of the tulip 200 may be preset or fixed prior to insertion into the patient.

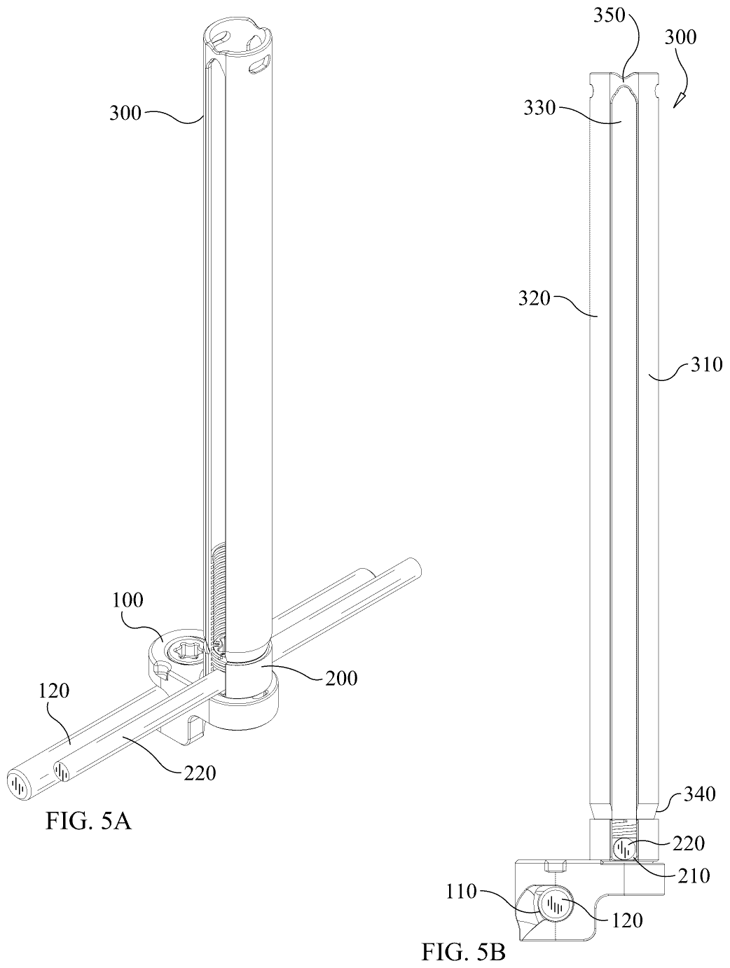

As shown for example in FIGS. 5A and 5B, 6 and 7, systems provided herein may include a main body 100 and a tulip 200 connected to the main body 100, and further a cannula 300 connected to the main body 100. In example embodiments the cannula may be connected to the tulip 200 prior to insertion into a patient, or it may be connected to the tulip 200 after insertion into a patient.

FIGS. 5A and 5B depict a representation of a working channel or cannula 300, created by two or more tabs 310 and 320 extending from the tulip 200 described herein. The tabs 310 and 320 may be detachable from the tulip 200 and/or from each other. A gap 330 between and defined by the tabs 310 and 320, is configured as a guide for the installation of the cross-connecting secondary rod 220. The gap 330 may extend from the slot 230 created by the tulip, such that the gap 330 and slot 230 are a contiguous opening. In example embodiments, the tabs 310 and 320 may be rigidly connected to the tulip 200 to create a temporary unthreaded sleeve for insertion into a patient, but can be cut when necessary.

In the examples shown in FIGS. 5A and 5B, which depict a configuration of a system having a main body 100, tulip 200 and cannula 300, as viewed from a perspective view (FIG. 5A) and from a side view (FIG. 5B), the first and second side openings 110 and 210, respectively, are aligned such that when primary and secondary rods 120 and 220 are inserted in the first and second side openings 110 and 210 respectively, the primary and secondary rods 120 and 220 extend in the same direction as one another, although at different heights than one another.

Notches 340 may be formed at a cannula 300-tulip 200 junction to facilitate detachment of the cannula 330/working channel from the tulip 200, upon application of bending or torsional loads by a user of the present systems (such as a surgeon). One or more notches 350 may be present in a junction between tabs 310 and 320 (and any optional additional tabs) that form the cannula 300, to enable or fascilitate separation of the tabs from one another.

While a single bodied, straight secondary cross connecting rod 220, with a circular cross section is shown in the systems of FIGS. 5A and 5B, it is contemplated that the second cross-connecting component may also be a plate, or may be a multi-bodied, curved, entailing circular, elliptical, rectangular, polygonal, triangular or a combination of the shapes in its cross-section.

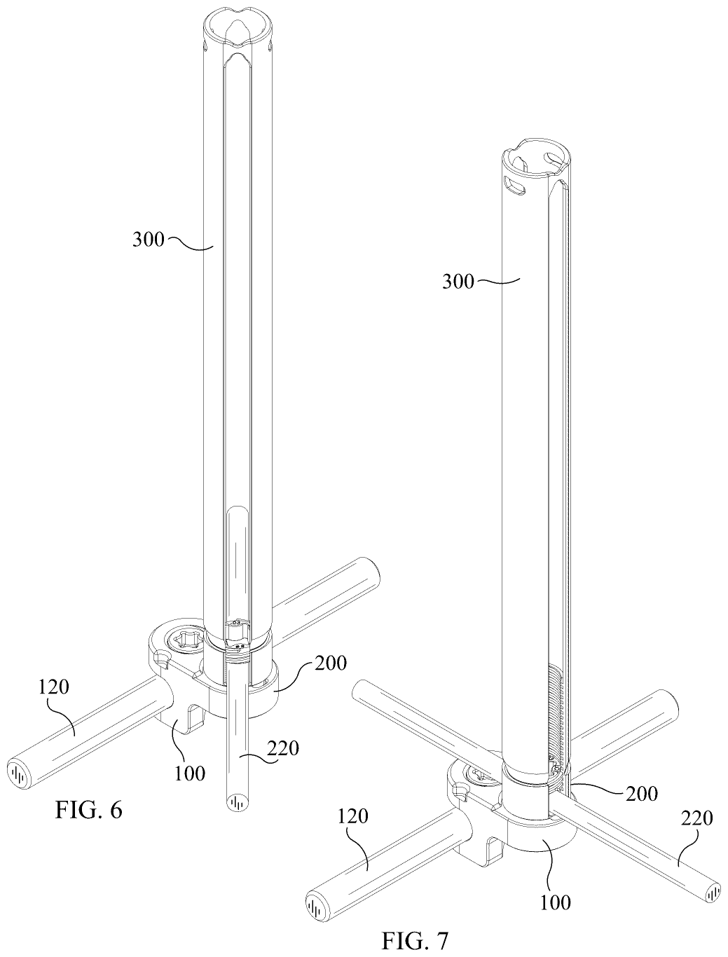

FIGS. 6 and 7 depict configurations of systems including a main body 100, tulip 200 and cannula 300, exhibiting different oblique angles that can be accomplished between the primary rod 120 and the secondary rod 220, for example in embodiments where the tulip 200 is in a rotated position with respect to the main body 100. In example embodiments, the primary and secondary rods 120 and 220, may cross each other in different planes, for example, with the secondary rod 220 over the primary rod 120. The oblique angles are not limited to those shown in FIGS. 6 and 7.

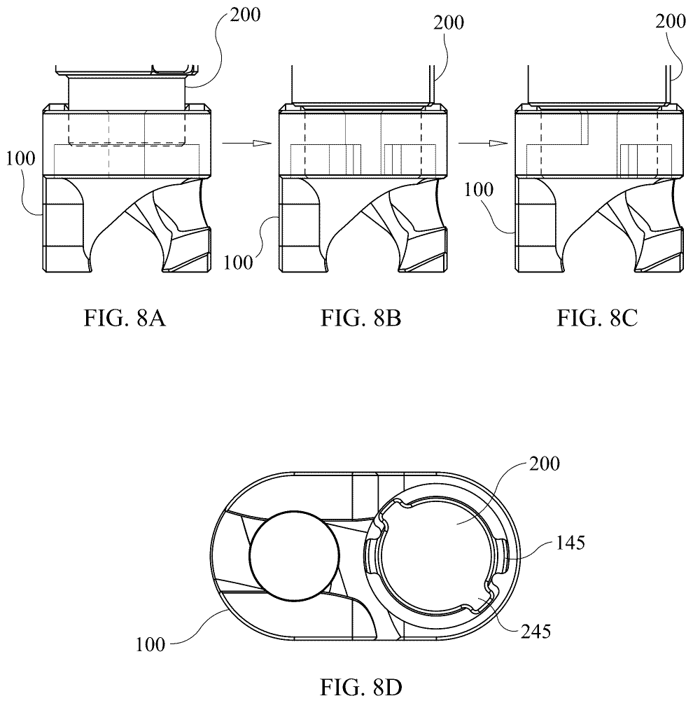

FIGS. 8A, 8B, and 8C show cross-sectional front views of a main body, for example as depicted in FIG. 1A, having a tulip 200 placed therein. FIGS. 8A-8C show a progression of placement of the tulip 200 within the second top opening 140 of the tulip. For example, FIG. 8A depicts the tulip 200 at an example first position of placement within the main body 100, FIG. 8B shows a placement of the tulip 200 at a second position of placement within the main body 100, in which a bottom portion 280 of the tulip is further within the main body 100. FIG. 8C shows a placement of the tulip 200 at a third position of placement within the main body 100, in which the tulip 200 is rotated within the main body 100.

In example embodiments, an external extrusion of the portal component mates with the internal shelf of the clamp/main body 100. FIG. 8A shows a portal component extrusion inserts axially/vertically into an internal gap 145 of the clamp component. FIG. 8B shows how, upon the application of relative torque between the portal and clamp 100 components, the extrusion on the portal component advances along the internal shelf of the clamp component. FIG. 8C shows a final position of the portal component where the internal shelf has a longer section that interferes with the portal component extrusion and halts its rotational progression.

FIG. 8D is a view of the main body 100 from above, having a tulip 200 therein, in which a top of the tulip 200 is shown with respect to the clamp 145 of the main body 100. In example embodiments, the clamp 145 of the main body 100 may have a female portion of an internal circumference that mates with a male portion 245 of an external circumference of the tulip. In a method of placing the tulip 200 within the main body 100, the tulip 200 may be rotated to a desired position. The tulip 200 may be rotated to be locked into the main body 100 for example, where the female portion of the clamp component 145 and the male component/extrusion 245 of the tulip 200 are aligned. Or in example embodiments, they may first be aligned and then a further rotation in either direction may occur, for example to lock the tulip 200 in the main body 100.

Components of the present devices may be made of various materials. According to example embodiments, components such as the main body, the tulip and/or a cannula may be a rigid or semi-rigid material for stability and/or to hold their shapes. For example, the tulip 200 and the main body 100 may be made of pure titanium or titanium alloys (for instance, titanium-aluminium-molybdenum alloy) thereof, stainless steel, Cobalt-Chromium, molybdenum rhenium or other materials known by those skilled in the art to be suitable implants. The various locking mechanisms, caps, rods, and other components described herein may also be of various same or different suitable materials.

Kits provided herein may include one or more of the system components described herein. For example, kits may include one or more of the following: a main body, a tulip, a cannula or component thereof, instructions for use of the present devices and systems, one or more rods or plates, such as the primary or secondary rods or plates, locking mechanisms and/or caps for use with the present devices and systems, or tools for assembly or insertion of the present devices or systems, or components thereof.

Also provided herein are methods of connecting spinal rods using the devices and systems provided herein. Non-limiting examples of the present methods may include the following, attaching the main body unto an existing rod, for example a rod already within a patient, attaching a locking mechanism, such as a screw 150 unto the main body, driving the screw to compress the existing primary rod 120, attaching the tulip 200 unto the main body 100 via a twist and lock mechanism, placing a secondary rod 220 into the tulip 200, adjusting the tulip and/or secondary rod to a desired angle, and driving the locking cap 240 into the tulip 200. In example embodiments, the locking cap 240 may be provided to the desired location through a cannula 300 including tabs 310 and 320 to compress/fixate the secondary rod, and the methods may further include detaching tabs 310 and 320 of the from each other and from the rest of the tulip. According to examples, methods may include attaching the other end of the secondary rod 220 to a pedicle screw or any other device designed to work with spine rods. The present methods may be minimally invasive methods that include utilization of a tulip with detachable tabs for visibility outside the patient's body and the placement of the secondary rod through the slot 330. Alternatively, methods may be provided for using the present systems and devices that are open methods.

By way of non-limiting example, the present methods may be used in MIS or open surgery methods on the spine, in all spine regions. For example, the present methods and devices may be used with respect to the thoracolumbar spine, or for cervical or sacral regions, but are not limited thereto.

The present devices systems and methods provide for example, top-loading of the main body, which allows a surgeon to place the device in an MIS or open surgery, and associated characteristics for placement and retention (such as undersized lips, for example, which may click for tactile feedback, and a retention feature, such as a screw, which compresses the main rod).

The present devices systems and methods also provide an ability for the tulip to be placed percutaneous simultaneously with the main body, or after the main body has been placed within a patient. The tulip may also be polyaxial with respect to the main body, for example, by means of the twist and lock mechanism, or they may be fixed with respect to each other.

The present devices systems and methods also provide an ability for percutaneous (MIS) or open surgery placement and retention of a secondary rod, due to the characteristics of the tulip, such as slots, threading for the locking cap 240, which retains/compresses the secondary rod 220, and optional extended tabs/petals 270, which can be separated from each other and/or broken off.

The present devices, systems, kits and methods provide a significant advancement in the field of minimally invasive spine surgery, addressing limitations of previous devices and methods, and potentially improving patient outcomes through enhanced construct stability and reduced surgical invasiveness.

While the present disclosure has been described in terms of exemplary aspects, those skilled in the art will recognize that the present disclosure can be practiced with modifications and/or variations within the spirit and scope of the application. The examples provided herein are merely illustrative and are not meant to be an exhaustive list of all possible designs, aspects, applications or modifications of the present disclosure

We claim:

1. A rod connector system comprising:
a main body defining a first side opening configured for insertion of a primary rod therein, and
a tulip defining a second side opening configured for insertion of a secondary rod therein, the tulip being inserted into a top opening in the main body and attached to the main body above the main body,
wherein the main body includes a locking mechanism for retaining the primary rod at a desired position therein, and
wherein the tulip includes a locking cap for maintaining the secondary rod at a desired position therein.

2. The rod connector system of claim 1, further comprising a cannula detachably attached to the tulip.

3. The rod connector system of claim 2, wherein the cannula comprises at least two tabs defining a channel therebetween.

4. The rod connector system of claim 1, wherein the main body further includes at least one lip configured to retain the primary rod.

5. The rod connector system of claim 4, wherein the at least one lip is configured to provide an audible or palpable confirmation of primary rod engagement to a desired location within the main body.

6. The rod connector system of claim 1, further comprising a relaxation slot at an end of a helical cut out in the main body configured to facilitate capture or placement of the primary rod in the main body.

7. The rod connector system of claim 1, wherein the tulip includes internal helical threads and the locking cap includes corresponding helical threads such that the locking cap is threadable in the tulip.

8. The rod connector system of claim 1, wherein the tulip is rotatable in a first direction with respect to the main body so the first side opening of the main body is at a desired angle with respect to the second side opening of the tulip.

9. The rod connector system of claim 1, wherein the main body and the tulip are rotatable with respect to one another such that the secondary rod in the tulip is parallel, perpendicular or at another angle with respect to the primary rod in the main body.

10. The rod connector system of claim 1, wherein the locking mechanism for retaining the primary rod at a desired position in the main body, includes a top loading screw.

11. The rod connector system of claim 1, wherein the tulip includes at least one petal configured to receive a cannula or tab therein.

12. A kit including the main body and the tulip of claim 1.

13. The kit of claim 12, further comprising a cannula or one or more tabs configured to form a cannula upon attachment to the tulip.

14. The kit of claim 12, further comprising at least one additional component selected from the group consisting of instructions for use of the system, at least one of the primary rod or the secondary rod, the locking mechanism, the locking cap, and a tool for assembly or insertion of the the rod connector system or a component thereof.

15. A method of connecting multiple spinal rods comprising
attaching a main body to a primary rod,
attaching a locking mechanism to the main body, the locking mechanism being configured to lock the primary rod to a desired position with respect to the main body by compressing the primary rod,
attaching a tulip to the main body,
placing a secondary rod into an opening of the tulip,
adjusting the tulip or the secondary rod, such that the secondary rod has a desired angle with respect to the primary rod, and
driving a locking cap into the tulip to lock the secondary rod at a desired position.

16. The method of claim 15, further comprising
attaching a cannula to the tulip configured to be suitable for inserting or guiding
the locking cap to the tulip.

17. The method of claim 16, wherein the cannula comprises one or more tabs that are configured to be detachable from each other and detachable from the tulip.

18. The method of claim 17, further comprising detaching the tabs from each other and from the tulip.

19. The method of claim 15, further comprising attaching the secondary rod to a pedicle screw.

20. The method of claim 15, wherein the method is an open or minimally invasive method.
